(19) United States
(12) Patent Application Publication
(10) Pub. No.: US 2014/0074147 A1
(43) Pub. Date: Mar. 13, 2014

(54) CLOT CAPTURE DEVICE

(71) Applicant: University of Miami, Miami, FL (US)

(72) Inventor: Eduardo De Marchena, Coral Gables, FL (US)

(73) Assignee: University of Miami Intellectual Property Strategy and Licensing IPSL, Miami, FL (US)

(21) Appl. No.: 13/698,707

(22) PCT Filed: May 23, 2012
(86) PCT No.: PCT/US12/39079

§ 371 Date: Nov. 19, 2012

Related U.S. Application Data
(60) Provisional application No. 61/489,028, filed on May 23, 2011.

(51) Int. Cl. A61F 2/01

(52) U.S. Cl. CPC A61F 2/01
USPC 606/200

(57) ABSTRACT

A system and method for preventing the movement of emboli away from a vascular region. The system generally includes a medical device having a clot capture assembly, a shaft, and a sheath disposed about the shaft, and a console. The clot capture assembly includes a first and second clot capture element, each of which is disposed between the shaft and sheath when in a first position. When the sheath is retracted, the first and second clot capture elements expand into a second position, at least a portion of the first and second clot capture elements being substantially in contact with at least a portion of an interior surface of a section of vasculature.

### FIELD OF THE INVENTION

The present invention relates to a method and device for preventing emboli from entering the carotid arties and capturing loosing emboli during a cardiovascular procedure.

### BACKGROUND OF THE INVENTION

Owing to advancements in surgical technology, the population of people undergoing cardiac surgery has significantly increased in the past two decades, as well as the age of the patients who are eligible to undergo such procedures. This older population is at a higher risk for age-related cerebrovascular disease, which may predispose them to severe neurological complications, including ischemic stroke and encephalopathy during the immediate postoperative period.

Medical procedures performed within a patient's vasculature may produce emboli (also referred to herein as blood clots or thromboemboli) that may travel through the blood stream from the treatment site to areas of smaller vascular passages, where they may lodge. Such a blockage may be fatal. For example, a brain embolism may cause a stroke.

Procedures performed in the heart may produce emboli that pass from the left ventricle of the heart into the aorta, from where they may travel into one of the carotid arteries leading to the brain and also the visceral and peripheral vascular systems. Patients may be given blood thinners to prevent clot formation, but these medications may have unintended side effects and create complications. Some patients may not be able to handle blood thinners, and therefore anticoagulation therapy may be a contraindication for extended periods of time. Another option is physically blocking access to the carotid arteries (brachiocephalic artery, left common carotid artery, and left subclavian artery) during procedures such as aortic valve replacement and other cardiac surgeries, which lowers the risk of emboli traveling through the vasculature and causing ischemic strokes. However, it is equally desirable to capture or prevent clots from traveling to downstream portions of the aorta, its tributaries (such as the common iliacs), and other regions of the vascular system.

Previous attempts to restrict access to the carotid arteries have included implanting stents with a portion of the aortic arch. However, such devices merely deflect and fail to capture emboli created during a surgical procedure, and as a result, patients are vulnerable to downstream complications from emboli. Other devices, such as baskets, have been developed to capture clots, but these devices are either difficult to deploy or are not configured to be left in place, even temporarily, when the procedure is over. Further, known devices fail to both deflect emboli from carotid arteries and capture emboli before they travel through the vascular system to other areas of the body. Still further, even if positioned over the ostium of one or more carotid arteries, known devices may not adequately conform to each patient's unique anatomy to fully block the carotid arteries. Still further, known devices do not provide a means for positioning a medical tool upstream of the device while preserving the deflection and capture functionality.

Therefore it is desirable to provide a device that both captures and deflects emboli to prevent embolic movement away from a treatment site throughout the vascular system. It is further desirable to provide a device that conforms to each patient's unique anatomy to prevent emboli from entering the carotid arteries. It is further desirable to provide a device that may be positioned within the vascular system that has a means for entry of a medical tool, such that the medical tool may perform a procedure upstream of the device while not compromising the deflection and capture functionality of the device.

### SUMMARY OF THE INVENTION

The present invention provides a system and method for preventing the movement of emboli away from a vascular region. The device generally includes a first expandable sieve element disposed within the medical device when in a first position, at least a portion of the first clot capture element defining a mesh, and a second expandable sieve element disposed within the medical device when in a first position, at least a portion of the second clot capture element defining a mesh, the second sieve element being positioned a distance away from the first sieve element.

The medical device may further include a sheath slidably disposed about the shaft. The first and second sieve elements may be disposed between the distal portion of the shaft and the sheath when in a first position. The first sieve element may have a distal portion and a proximal portion, with the distal portion having a diameter that is greater than the proximal portion when the first sieve element is in a second position. Further, the second sieve element may have a substantially tubular shape when in a second position. The second sieve element may further include a support element and a stabilization element. Further, the mesh may define a plurality of openings having a diameter of between approximately 80-100 microns.

The method may generally include advancing into a mammalian aorta a medical device including a shaft having a distal portion, a sheath disposed about the distal portion of the shaft, and a clot capture assembly disposed between the distal portion of the shaft and sheath when in a first position, retracting the sheath from the distal portion of the shaft, the distal assembly transitioning to a second expanded position, performing a medical procedure on the vascular region, and retracting the clot capture assembly within the medical device. The clot capture assembly may include a first and second clot capture element, at least a portion of each of the first and second clot capture elements being substantially in contact with an inner surface of the aorta and at least one carotid artery ostium.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows a system including a medical device having a clot capture assembly;

FIG. 2 shows a perspective side view of an expanded clot capture assembly including a first and second clot capture element;

FIG. 3 shows a top view of a first clot capture element having a device access area;

FIGS. 4A-4B show perspective side views of a first mechanism for clot capture assembly deployment;

FIGS. 5A-5B show perspective side views of a second mechanism for clot capture assembly deployment; and

FIGS. 6-8 show a sequence of expansion of the clot capture assembly.

### DETAILED DESCRIPTION OF THE INVENTION

The term “clot capture assembly” is used herein to refer to an assembly that may include a first and second clot capture element that are expandable from a medical device into an area of vasculature (for example, the aorta).

The term “clot capture element” is used herein to refer to an element, mechanism, or device that captures, deflects, or otherwise prevents the spread of clots (or emboli) from the vasculature in which the clot capture device is positioned. For example, the coronary artery bypass graft procedure may produce emboli that may travel throughout the patient's body and lead to ischemic stroke. The clot capture device may either physically retain (capture, such as by a sieve) the emboli, deflect the emboli from entering, for example, the carotid arteries leading away from the aortic arch, or otherwise prevent the spread of emboli within the patient's body.

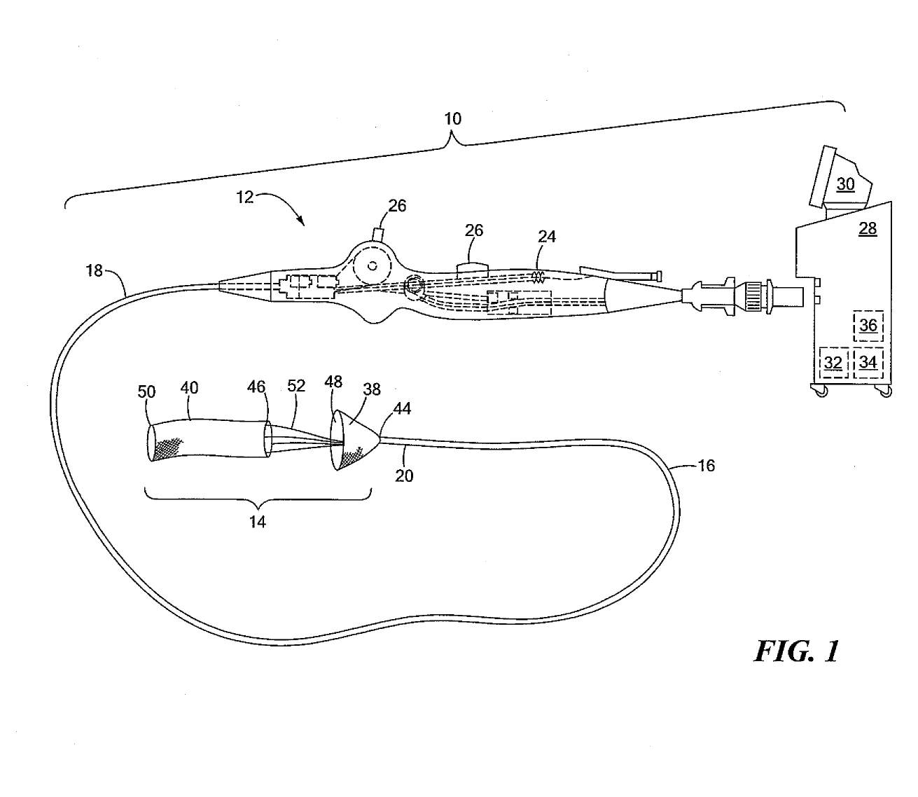

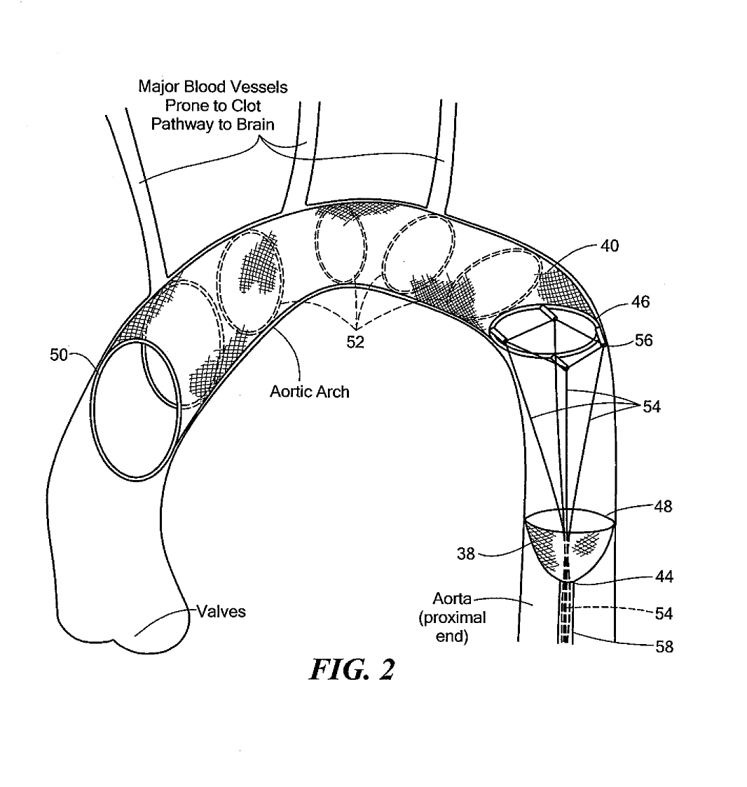

Now referring to FIG. 1, a system including a medical device having a clot capture assembly is shown. The system 10 generally includes a medical device 12 having a clot capture assembly 14 (as shown and described in FIGS. 2-8) and a flexible elongate body or shaft 16 having a proximal portion 18, a distal portion 20, and one or more lumens 22 defined therebetween. The distal portion 20 of the shaft 16 may be more or less flexible than the proximal portion 18, or the shaft may have the same flexibility throughout. The medical device 12 may further include a handle 24, which may include one or more actuators 26 (such as knobs, levers, and the like), couplings, fluid and/or electrical ports, and other such features. The system 10 may also include a console 28 in communication with the distal portion 20 of the shaft 16 and/or the clot capture assembly 14. The console 28 may include a computer 30 having one or more displays and user interfaces for monitoring and adjusting system parameters, a power source 32, and other elements such as a cryogenic fluid reservoir 34 and/or energy generator 36. For example, if the medical device 10 is a radio frequency (RF) ablation catheter, the console will include an RF generator. Additionally or alternatively, any of a number of secondary devices may be used for the medical procedure (for example, an RF, cryo, ultrasound, laser, or microwave ablation catheters), these secondary devices being in communication with the console 28. However, the system 10 may not include a console 28, instead being operable with a medical device 12 having a clot capture assembly 14, flexible elongate body or shaft 16, and a handle 24.

The system 10 may further include a sheath 37 through which the medical device and/or secondary devices may be advanced to a predetermined location within the vasculature. The sheath 37 may define one or more lumens for removable insertion of medical devices such as secondary devices. Exemplary surgical devices may include radiofrequency, cryogenic, laser, microwave, ultrasound, and the like energy transfer catheters, micro scissors, or other devices operable to perform surgery on the aortic valves and the surrounding region.

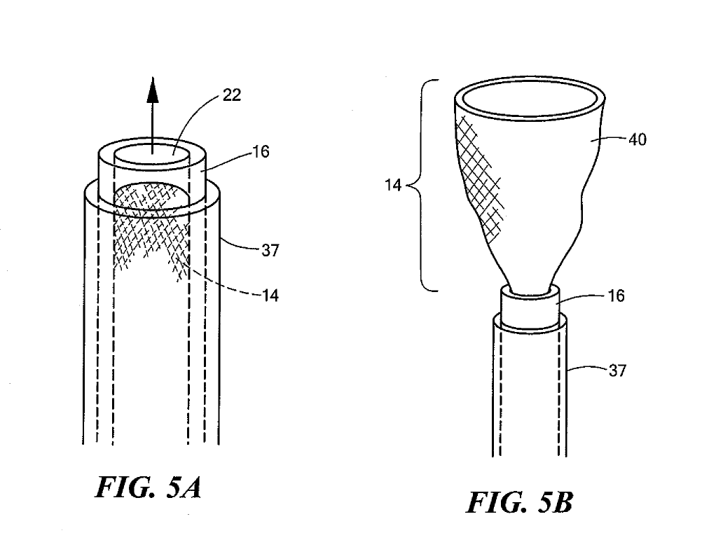

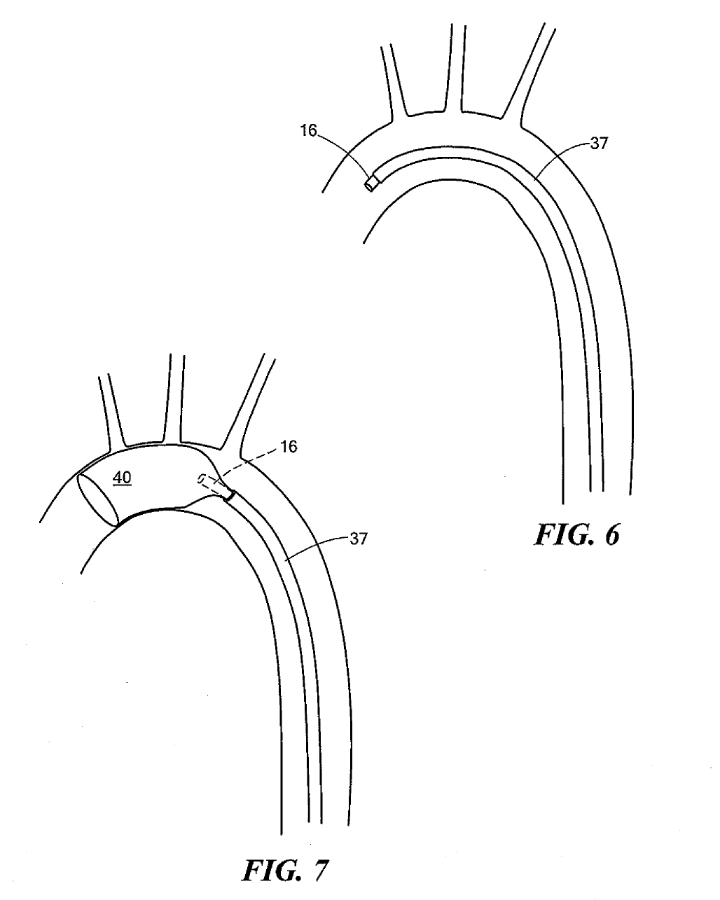

Now referring to FIG. 2, an expanded clot capture assembly 14 including a first clot capture element 38 and second clot capture element 40 is shown. In this figure, the medical device 12 is positioned within an area of the patient's vasculature, such as the aorta. For example, a sheath 37 may first be advanced into the aorta, and the medical device 12 advanced through the sheath. The clot capture assembly 14 may include a first clot capture element 38 and a second clot capture element 40, both being releasable or deployable together from the distal portion 20 of the shaft 16 (as shown and described in FIGS. 4A-8). Further, the first and second clot capture elements 38, 40 each have a proximal portion 44, 46 and a distal portion 48, 50. The second clot capture element 40 may substantially line (that is, be in contact with) the inner circumference of an area of vasculature in order to deflect emboli into the first clot capture element 38. As shown in FIG. 2, if positioned within the aorta (such as the aortic arch), the second clot capture element 40 may also deflect emboli into the first clot capture element 38, thereby preventing emboli from traveling into the carotid arteries connected to the aorta. The first and second clot capture elements 38, 40 are shown and described in more detail in FIGS. 4A-5B.

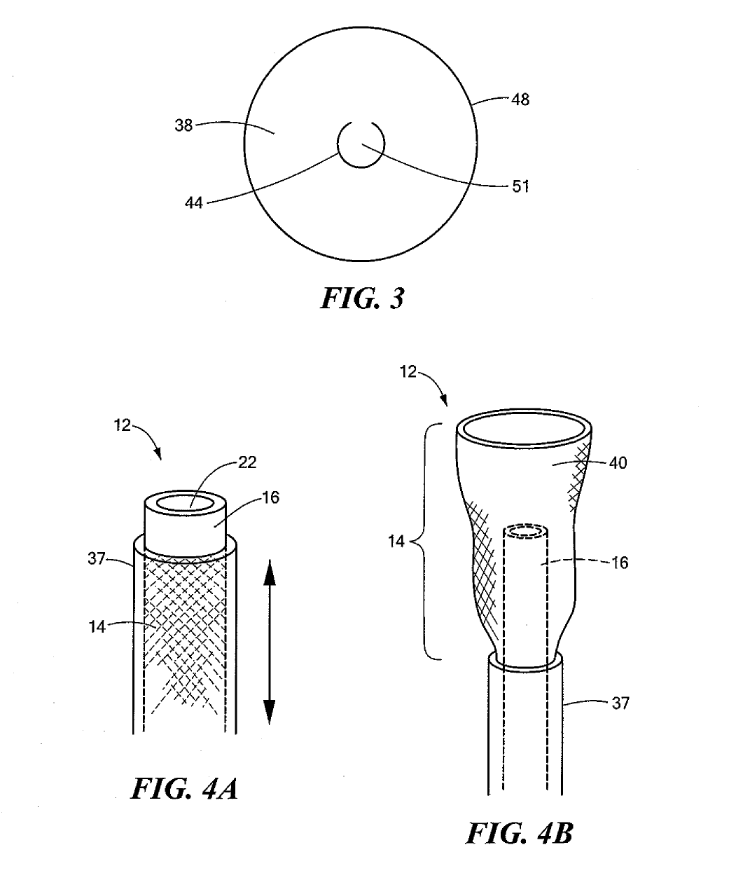

The first clot capture element 38 when expanded may assume a funnel- or cone-like shape (as shown in FIGS. 1 and 2). Further, the first clot capture element 38 may be open at the distal portion 48 and closed or substantially closed at the proximal portion 44 in order to capture emboli traveling through the bloodstream. Further, the distal portion 48 may be substantially in contact with at least a portion of the interior surface of the vascular in which the device 12 is positioned in order to prevent the unintended escape of emboli between the vasculature inner wall and the clot capture element 38 (as shown in FIG. 2). The first clot capture element 38 may further include an assembly entry area 51 or means for entry of a medical tool or device through the first clot capture element 38 (as shown and described in FIG. 3). The assembly entry area 51 allows a medical device to enter the clot capture assembly 14 through the first clot capture element 38 and be positioned upstream of the clot capture assembly 14 to perform a medical procedure without compromising the deflection and capture functionality of the clot capture assembly 14.

The second clot capture element 40 when expanded may assume a substantially cylindrical or tubular shape that is open at the ends of both the proximal 46 and distal 50 portions, being sized to conform to and span an area of vasculature (for example, the aortic arch proximate the proximal ends of the carotid arteries). The second clot capture element 40 may assume the contours of the anatomical area, such as the curve of the aortic arch (as shown in FIG. 2). Although the second clot capture element 40 may not capture emboli as does the fist clot capture element 38, the second element 40 may instead deflect emboli away, for example, from the carotid arteries and into the first element 38. As an example, if a medical procedure is conducted within or proximal to the heart, emboli may be produced that travel from the procedure site and through the aorta. The emboli may first travel through the second clot capture element 40, thereby being prevented from entering the carotid arteries, and then travel into the first clot capture element 38 wherein they become trapped.

The second clot capture element 40 may further include a plurality of scaffolding elements 52 contouring and connected to the inner or outer surface of the circumference of the second clot capture element 40. The scaffolding elements 52 may be composed of a self expanding metal such as Nitinol, plastics, or other materials that provide suitable structural support and are operable to apply pressure to the clot capture element 40 such that the outer surface of the clot capture element 40 remains substantially in contact with the inner wall of the vasculature (such as the aorta). That is, each scaffolding element 52 may be biased toward an expanded position, thus exerting outward force on the inner wall of the vasculature. Each scaffolding element 52 may be independently expandable. Thus, the second clot capture element 40 with scaffolding elements 52 may conform to a patient's unique anatomy. Any number of scaffolding elements 52 may be provided and may be spaced variably or uniformly from each other. As an example, each of the scaffolding elements 52 may expand to conform to the shape of the inner surface of the aorta. Alternatively, one or more scaffolding elements 52 may be included, each expanding to form a substantially helical shape.

Each of the first and second clot capture elements 38, 40 may define a pattern (such as a mesh) on at least a portion of their outer surface that has a porosity, for example, approximately 80-100 Microns in diameter, to prevent clots from traveling away from the area of the medical device 12 (for example, prevent clots from entering the carotid arteries and/or deflect clots ascending from the aorta), but allow blood to flow through freely. Further, the clot capture elements 38, 40 may be composed of a biocompatible and deformable material such as a nylon or Nitinol that may be expanded by an actuator in communication with the medical device 12, or may expand automatically to a predetermined shape, for example, a basket, in response to an increase in temperature. The material of the clot capture elements 38, 40 should be such that it will not damage the patient's vasculature when the elements 38, 40 are expanded. Further, each of the clot capture elements 38, 40 may be biased in either the expanded or retracted position.

The clot capture assembly 14 may include a stabilization element for connecting the first and second clot capture elements 38, 40 and for facilitating retrieval of the clot capture assembly 14. For example, the stabilization element may operate to maintain the axial position of the second clot capture element 40 during a surgical procedure. The stabilization element also functions to space the first and second clot capture elements 38, 40 a distance apart when deployed. The stabilization element may include a plurality of tethers or wires 54 coupled to the proximal portion 46 of the second clot capture element 40 at a plurality of coupling locations 56. The tethers 52 may be flexible, and may further be coupled to the distal 48 and/or proximal 44 portions of the first clot capture element 38. Alternatively, the tethers 52 may run through a small opening in the proximal portion 44 of the first clot capture element 38 (such as the assembly entry area 56 shown in FIG. 3). Additionally, the clot capture assembly 14 may include a retrieval wire 58 coupled to the proximal portion 44 of the first clot capture element 38 to aid in retrieval of the assembly 14.

Now referring to FIG. 3, an assembly entry area is shown. The assembly entry area 51 allows a medical device to enter the clot capture assembly 14 through the first clot capture element 38 and be positioned upstream of the clot capture assembly 14 to perform a medical procedure without compromising the deflection and capture functionality of the clot capture assembly 14. For example, when the clot capture assembly 14 is deployed within, for example, the aorta, the shaft 16 and sheath 37 may be withdrawn from the vasculature. The clot capture assembly 14 may be left in place until later retrieval (for example, the assembly 14 may remain in place for two hours). However, it may be necessary to reinsert a sheath 37 and/or medical device through the clot capture assembly 14 in order to retreat or perform a subsequent medical procedure in the heart or areas of the vasculature that are upstream of the assembly 14. Therefore, the first clot capture element 38 may include an assembly entry area 51 through which the sheath 37 or medical device may pass.

FIG. 3 shows a top view of the first clot capture element having an assembly entry area with a “trap door” type closure. The closure of FIG. 3 may be coupled to the proximal portion 44 of the first clot capture element 38 by only a small part of the closure. The flow of blood into the first clot capture element 38 will keep the assembly entry area 51 closed, but an upward (that is, toward the distal portion 48 of the clot capture element 38) force exerted by a sheath or medical device will force open the closure, thereby allowing sheath or device to pass through the assembly 14 to areas of the vasculature that are upstream of the assembly 14. Emboli trapped within the first clot capture element 38 may pass into a lumen of the entering medical device, but will not escape the assembly 14 into the patient's vasculature. Thus, the assembly entry area 51 may include a closure that only opens in one direction. Alternatively, an iris-type closure may be used (not shown), wherein the iris-type closure is opened only to an extent that allows entry of the medical device while preventing embolic escape.

Now referring to FIGS. 4A-5B, mechanisms for deployment of the clot capture assembly 14 are shown. As shown in FIG. 3A, a slidably movable sterile sheath 37 may surround (be disposed about) at least a portion of the distal portion 20 of the shaft 16 to compress the clot capture assembly 14 in a retracted or folded position against the shaft 16. When the sheath 37 is retracted, using either a manually or electrically operated actuator, the clot capture assembly 14 may expand from a folded or retracted position (first position, as shown in FIG. 4A) into an unfolded or expanded position (second position, as shown in FIG. 4B). For example, in the folded position, the clot capture assembly 14 may be disposed between the sheath 37 and the shaft 16. As shown in FIG. 4B, the second clot capture element 40 may be exposed before the first clot capture element 38 (for example, the first clot capture element 38 is not yet exposed in FIG. 4B). The distal portion of the sheath 37 may be funnel-shaped, with the distal end of the funnel shape having a greater diameter than the proximal end of the funnel shape, to facilitate deployment of the assembly 14 (not shown)

As shown in FIG. 5B, the clot capture assembly 14 may alternatively be disposed within and released from the lumen 22 of the medical device 12. In this case, although a sheath may be used to facilitate placement of the medical device 12, it may not play a role in deployment of the clot capture assembly 14. As non-limiting examples, the clot capture assembly may be pushed through the lumen 22 and out of the distal portion 20 of the shaft 16 by a rod or similar device slidably disposed within the lumen 22 (not shown). Alternatively, a guidewire may be coupled to the assembly 14, wherein advancing the guidewire through the lumen 22 and out the shaft 16 with bring the clot capture assembly 14 with it (not shown).

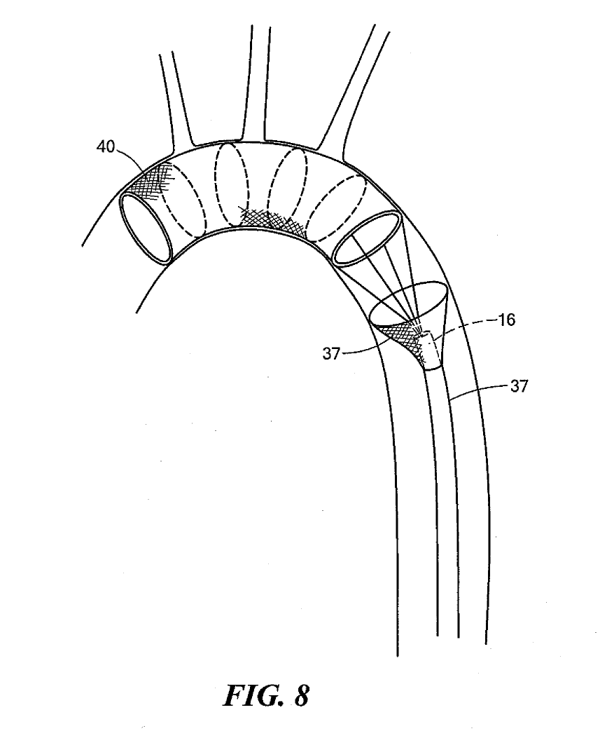

Now referring to FIGS. 6-8, a sequence of clot capture assembly 14 deployment is shown. In FIG. 6, the sheath 37 and shaft 16 are advanced through the vasculature to a predetermined location, for example, in the aorta at or proximal to the aortic arch, upstream of the carotid arteries. The medical device 12 may include a guidewire (not shown) slidably disposed within the main lumen 22 or other lumen of the device 12, and may be used to facilitate navigation through and/or positioning within the vasculature. The target or desired position of the medical device 12 may be determined through, for example, fluoroscopy or other positioning systems, to increase the accuracy of the clot capture element 38, 40 deployment.

The clot capture assembly shown in FIG. 7 is disposed between the sheath 37 and the shaft 16 (as shown and described in FIGS. 4A and 4B). The sheath 37 and/or medical device 12 may be retreated mechanically or electrically by an actuator (for example, toward the proximal portion of the aorta), and this retraction allows for the expansion or unfolding of the clot capture element assembly. As shown in FIGS. 4B, 5B, and 7, the second clot capture element 40 may be exposed from the device 12 before the first clot capture element 38. The scaffolding elements 52 and the second clot capture element 40 transition from a first position (folded or compressed within the shaft) to a second expanded position, contacting the inner wall of the aorta (as shown in FIG. 8). As the sheath 37 continues to retract, the stabilizing element (for example, a plurality of tethering elements 54) may be exposed toward a proximal end of the aorta (as shown in FIG. 8). The final appearance of the device 12 when in position may be as shown and described in FIG. 2.

When the clot capture assembly 14 is expanded, the physician may slidably insert a surgical device through the sheath 37, as discussed above, to perform a surgical procedure on the aorta or aortic valves. Additionally or alternatively, a device may be inserted through the assembly 14 at a later time (as shown and described in FIG. 3). During the procedure clots may be captured and/or deflected by the clot capture assembly 14.

To retrieve the assembly 14, the physician may retract the medical device 12 within the sheath 37 and/or slide the sheath 37 forward to collapse and/or retract the first clot capture element 38, stabilization elements 52, and the second clot capture element 40 within the shaft. To facilitate recapture, the sheath 37 may include a funnel-like distal end that compresses the assembly 14 within the device 12 as the sheath 37 is advanced and/or the device 12 is retracted. If the assembly 14 has been left in place and the medical device 12 and/or sheath has been removed from the area, the medical device 12 and sheath 37 may be reintroduced and one or more guidewires, hooks, or other means may be used to capture the one or more stabilization elements 52 (for example, tethers 54) and/or retrieval wire 58 and pull the assembly 14 into the sheath 37 and/or medical device 12. Owing to the orientation and position of the clot capture elements 38, 40, captured emboli are retained and enclosed by, for example, a trawling-like process, which prevents emboli from escaping. Additionally, the clot capture elements 38, 40 may be coated with a material having a low coefficient of friction, so that as the clot capture assembly 14 is expanded or retracted, the production of emboli or irritation of the aorta is minimized. The medical device 12 may be safely removed from the patient, bringing with it any captured emboli.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. A medical device comprising:
a first expandable sieve element disposed within the medical device when in a first position, at least a portion of the first sieve element defining a mesh; and
a second expandable sieve element disposed within the medical device when in a first position, at least a portion of the second sieve element defining a mesh, the second sieve element positioned a distance away from the first sieve element.

2. The device of claim 1, further comprising a shaft having a distal portion, a proximal portion, and defining one or more lumens therebetween.

3. The device of claim 2, further comprising a sheath slidably disposed about the shaft.

4. The device of claim 3, wherein the first sieve element is disposed between the distal portion of the shaft and sheath when in the first position, the first sieve element transitioning from the first position to a second position when the shaft is retracted from the distal portion of the shaft.

5. The device of claim 4, wherein the first sieve element has a distal portion and a proximal portion, the distal portion having a diameter that is greater than the proximal portion when the first sieve element is in a second position.

6. The device of claim 3, wherein the second sieve element is disposed between the distal portion of the shaft and sheath when in the first position, the second sieve element transitioning from the first position to a second position when the shaft is retracted from the distal portion of the shaft.

7. The device of claim 6, wherein the second sieve element has a substantially tubular shape when in the second position, the second sieve element being substantially in contact with an inner surface of a length of vascular tissue when in the second position.

8. The device of claim 5, wherein the first sieve element includes a closure element in the proximal portion, the closure element being openable in one direction.

9. The device of claim 8, wherein the vascular tissue is the aorta.

10. The device of claim 9, wherein the vascular tissue is the aortic arch, the second sieve element further being in contact with the ostium of at least one carotid artery.

11. The device of claim 6, further including a stabilization element coupled to the second sieve element.

12. The device of claim 11, wherein the stabilization element includes a plurality of tethers.

13. The device of claim 8, further including a support element coupled to the second sieve element, the support element exerting an outward pressure keeping the second sieve element substantially in contact with the inner surface of the length of vascular tissue when the second sieve element is in the second position.

14. The device of claim 1, wherein the mesh defines a plurality of openings, each opening having a diameter of between approximately 80-100 microns.

15. A medical device comprising:
a shaft having a proximal portion, a distal portion, and one or more lumens extending therethrough;
a sheath slidably disposed about at least a portion of the distal portion of the shaft;
a first clot capture element; and
a second clot capture element, the first and second clot capture elements each defining a mesh and being disposed between the shaft and sheath when in a first position.

16. The device of claim 15, wherein the first and second clot capture elements each transition from the first position to a second position when the shaft is retracted from the distal portion of the shaft.

17. The device of claim 16, wherein the first clot capture element has a distal portion and a proximal portion, the distal portion having a diameter that is greater than the proximal portion and the distal portion being substantially in contact with at least a portion of an interior surface of the aorta when the first clot capture element is in the second position.

18. The device of claim 17, wherein the second clot capture element has a substantially tubular shape when in the second position, the second clot capture element being substantially in contact with at least a portion of an anterior surface of the aorta and at least one of the carotid artery ostia when the second clot capture element is in the second position.

19. A method of preventing movement of emboli from a vascular region, comprising:
advancing into a mammalian aorta a medical device including a shaft having a distal portion, a sheath disposed about at least a portion of the distal portion of the shaft, and a clot capture assembly disposed between the distal portion of the shaft and sheath when in a first position;
retracting the sheath from the distal portion of the shaft, the distal assembly transitioning to a second expanded position;
performing a medical procedure on the vascular region; and
retracting the clot capture assembly within the medical device.

20. The method of claim 19, wherein the clot capture assembly includes a first and second clot capture element, at least a portion of each of the first and second clot capture elements being substantially in contact with an inner surface of the aorta and at least one carotid artery ostium.
